(19) United States
(12) Patent Application Publication
(10) Pub. No.: US 2007/0122463 A1
(43) Pub. Date: May 31, 2007

(54) ANTIMICROBIAL COMPOSITIONS AND WOUND DRESSINGS

(75) Inventor: Tse-Hao Ko, Taichung (TW)

Correspondence Address: PATTERSON, THUENTE, SKAAR & CHRISTENSEN, P.A., 4800 IDS CENTER, 80 SOUTH 8TH STREET, MINNEAPOLIS, MN, 55402-2100, US

(73) Assignee: FENG CHIA UNIVERSITY, Taichung (TW)

(21) Appl. No.: 11/562,284

(22) Filed: Nov. 21, 2006

(30) Foreign Application Priority Data
Nov. 25, 2005 (TW) 094141437
Sep. 19, 2006 (TW) 095134585

(51) Int. Cl. A61K 33/44; A61K 33/38; A61K 31/7048; A61K 31/7034; A61K 31/65; A61K 31/545; A61L 15/00; A61K 36/28

(52) U.S. Cl. 424/445; 424/618; 424/125; 514/29; 514/35; 514/152; 424/764; 514/200

(57) ABSTRACT

A wound dressing and an antimicrobial composition are provided. The wound dressing comprises a carbonaceous material, a noble metal carried on the carbonaceous material, and, optionally, a flexible base layer, wherein the carbonaceous material is selected from a group consisting of activated carbon, graphite, carbon, and a combination thereof, while the noble metal is selected from a group consisting of Ag, Au, Pd, Pt, Cu, Zn, and a mixture thereof. The antimicrobial composition is for topical use on skins and comprises the aforementioned carbonaceous material as well as the noble metal carried on the carbonaceous material.

This application claims priorities to Taiwan Patent Application No. 094141437 filed on Nov. 25, 2005 and 095134585 filed on Sep. 19, 2006.

### CROSS-REFERENCES TO RELATED APPLICATIONS

Not applicable.

### FIELD OF THE INVENTION

The present invention relates to the use of a noble metal carried on a carbonaceous material in an antimicrobial composition and an antimicrobial wound dressing. The present invention especially relates to an antimicrobial composition and a wound dressing containing a noble metal carried on an activated carbon or porous graphite. In particular, the present invention actively aggregates and destroys the microbes close to said composition and wound dressing to prevent microbial infection.

### BACKGROUND OF THE INVENTION

In the past, antimicrobial drugs were applied directly onto the injured portion of the body. Recently, depending on the type of wound, medical wound dressings, such as medical tapes, patches, gelatin film bandages and band-aids, have been adopted to cover the wound. The wound is thus isolated and protected from improper contact with the external atmosphere, thereby preventing infection and reducing pain.

It is known that noble metals, such as silver, gold, palladium, platinum, copper, and zinc, in dressings effectively kills microbes. The use of such noble metals in dressings also has been disclosed in many references. For example, Robert Edward Burrell et al. (U.S. Pat. No. 6,692,773 B2) disclose the use of a noble metal such as silver, gold, palladium, or platinum, or an alloy or a compound thereof, in a nanocrystalline form with a grain size of less than 100 nm, in coating a fabric to provide a dressing having the antiproliferative effect. Widemire (U.S. Pat. No. 5,782,788) discloses the fixation of a silver foil layer on a gauze pad to inhibit the growth of bacteria, virus, and fungus. Fabo (U.S. Pat. No. 5,340,363) discloses a dressing comprising an outer absorbent layer and an inner porous hydrophobic layer knitted of elastic threads and encapsulated by a soft hydrophobic silicone or polyurethane gel, wherein the gel can be used as a carrier for an antibacterial agent (e.g., zinc), a pain-relieving substance, and an agent that stimulates wound repair.

Klippel et al. (U.S. Pat. No. 3,830,908) disclose the use of micronized allantoin as a carrier for a bactericidal or bacteriostatic ingredient (such as silver citro allantoinate). The composition is dispersed on the surface of a plastic air splint or other bandaging product to provide antibacterial action, depending on the molecular dissociation. McKnight et al. (U.S. Pat. No. 3,800,792) disclose a surgical dressing comprising a layer of tanned and reconstituted collagen foam film, which is laminated to a thick continuous layer of an inert polymer. The collagen layer contains a finely-divided silver metal added by soaking the collagen film in Tollen's reagent. Stowasser (U.S. Pat. No. 2,934,066) discloses a dressing of absorbent metal-coated fibers, such as a carding fleece coated with aluminum and backed by compressed cellulose, and polyamide fibers coated with vacuum-deposited silver.

Given the prior technology mentioned above, it is clear that antimicrobial noble metals are widely used in healing wounds, especially in dressings. However, the use of these noble metals still focuses on the noble metal alone and at most, the noble metal in a form of an alloy or compound. There is no disclosure concerning the use of a noble metal carried on a carbonaceous material. Moreover, although the prior use can provide an antimicrobial effect, the microbes are only destroyed upon “coincidentally” coming into contact with the noble metal. Because of this, the noble metals merely provide a “passive” antimicrobial effect.

Moreover, as known by persons with ordinary skill in the art, the above-mentioned noble metals can only fight microbes in the presence of moisture. Therefore, for practical use, the medium must always be moist. If necessary, water is added to provide a moist environment. For example, according to U.S. Pat. No. 6,692,773 B2, when a nanocrystalline noble metal is coated onto a dressing for topical use on skin, the dressing must maintain a moist condition for the noble metal to exhibit the desired efficacy (see column 6, line 64 to column 7, line 17). Consequently, U.S. Pat. No. 6,692,773 B2 further discloses the preference for an absorbent layer in the dressing to hold in moisture for activating the noble metal (see said patent, column 5, lines 33 to 37). Such need of moisture for activating noble metals can also can be found in other references, such as the content published via the website, http://www.burnsurgery.org/Modules/silver/images/section7b/Mid_partial_thickness3copy.jpg.

The present invention relates to another format of the therapeutic application of a noble metal that has an antimicrobial effect. In a preferred embodiment, the present invention can actively aggregate and then destroy microbes so as to effectively reduce, and even prevent, microbial infection.

### SUMMARY OF THE INVENTION

According to the present invention, the carbonaceous material mentioned herein can be in the form of a powder, particle, fiber or sheet unless otherwise specified.

One object of the present invention is to provide a wound dressing comprising a carbonaceous material, a noble metal carried on a carbonaceous material, and, optionally, a flexible base layer. The carbonaceous material is selected from a group consisting of activated carbon, graphite, carbon, and a combination thereof, and the noble metal is selected from a group consisting of sliver, gold, palladium, platinum, copper, zinc, and a combination thereof. The dressing can optionally comprise other therapeutically active component(s).

Another object of the present invention is to provide a kit comprising the wound dressing of the present invention and a fixing element. The fixing element can be any device that fixes the wound dressing to the skin. For example, the fixing element could be a tape, a bandage, or a patch.

A further object of the present invention is to provide an antimicrobial composition for topical use on skin comprising a noble metal carried on a carbonaceous material, wherein the carbonaceous material is selected from a group consisting of activated carbon, graphite, carbon, and a combination thereof, and the noble metal is selected from a group consisting of sliver, gold, palladium, platinum, copper, zinc, and a combination thereof. Said composition can optionally comprise other therapeutically active components.

In the present invention, the use of an activated carbon or porous graphite as the carbonaceous material is preferred. It is believed that the activated carbon/porous graphite can aggregate and absorb microbes on its surface due to the electrostatic interactions or van der Waal forces between the microbes and the surface of the activated carbon/porous graphite, as well as the bioaffinity of the activated carbon/porous graphite. Consequently, as compared with the passive effect of the prior art in which microbes are destroyed and killed only when “coincidentally” coming into contact with the noble metal, the embodiments of the present invention adopt an active manner in aggregating and destroying the microbes around the noble metal to provide a superior antimicrobial effect. Moreover, in an embodiment that utilizes activated carbon as the carbonaceous material, there is no need for adding moisture. In other words, as compared with the prior art that requires moisture and/or a moisture-holding component, such as an absorbent layer, to activate the antimicrobial noble metal, the present invention provides a simpler way to attain the desired antimicrobial benefit.

### BRIEF DESCRIPTION OF THE DRAWINGS

FIG. 1 shows a schematic diagram of an embodiment of the wound dressing of the present invention, wherein the wound dressing is a patch.

FIG. 2 shows a schematic diagram of another embodiment of the wound dressing of the present invention, wherein the wound dressing is a patch.

FIG. 3 shows a schematic diagram of a further embodiment of the wound dressing of the present invention, wherein the wound dressing is gauze. FIG. 3A shows a partially enlarged top view of the gauze.

FIG. 4 shows a schematic diagram of another further embodiment of the wound dressing of the present invention, wherein the wound dressing is a band-aid.

FIG. 5 shows a cross-sectional view along line 5-5 in FIG. 4.

### DESCRIPTION OF THE INVENTION INVENTION

The wound dressing of the present invention comprises a carbonaceous material, a noble metal carried on the carbonaceous material and, optionally, a flexible base layer. The flexible base layer comprises a first surface and a second surface. The noble metal with carbonaceous material is applied onto at least one portion of said first surface.

The flexible base layer in the wound dressing of the present invention can be porous or drug-impermeable. If a gauze or band-aid is desired, the flexible base is suitably composed of a porous material, preferably of a non-adhesive material. If a patch is desired, it is suitable to adopt a drug-impermeable base. Materials that are conventionally used for providing a wound dressing and that also meet the above porous or drug-impermeable requirement can be used in the present invention. The porous base can be prepared from, for example, but not limited to, a woven fabric or a non-woven fabric, composed of one or more the following materials: natural fiber, polyolefin fiber, polyester fiber, polyurethane fiber, polyamide fiber, polycellulose fiber, and cotton fiber. Non-woven fabric is preferred. Polymers suitable for providing a drug-impermeable base comprise polyvinyl chloride, polyvinyl dichloride, polyolefin (e.g., ethylene vinylacetate copolymer, polyethylene, and polypropylene), polyurea, and polyester (e.g., polyethylene terephthlate). The drug-impermeable base can be a single polymer layer or film, or a laminate composed of multiple polymer layers.

The carbonaceous material in the wound dressing of the present invention is selected from a group consisting of activated carbon, graphite, carbon, and a combination thereof. Activated carbon or porous graphite is preferred. For example (but not limited to), activated carbon fiber, particulate or powder charcoal, porous graphite such as flexible graphite or extended graphite, and/or sheets obtained from the compression of powder and/or particulate activated carbon and/or porous graphite can be utilized. The noble metal is selected from a group consisting of sliver, gold, palladium, platinum, copper, zinc, and a combination thereof, preferably silver.

Any suitable means, such as immersing, thermocracking, electroplating, electroless plating, or vacuum plating, can be used to carry the noble metal on the carbonaceous material. Moreover, the noble metal can be carried on the carbonaceous material in any known form. To effectively release the noble metal ion that has antimicrobial activity, it is preferred for the noble metal particulate on the carbonaceous material to be no more than 200 nm, preferably no more than 100 nm, and most preferably no more than 50 nm.

The preparation of a silver-carrying carbonaceous material is provided as an example for illustrating several processes which can be used in preparing a noble metal-carrying carbonaceous material useful in the present invention:

Manner I:

a. Immersing in a Solution of Silver Nitrate

An activated carbon fiber is immersed in a solution of silver nitrate for 1 to 720 minutes to reduce the silver on the surface of the activated carbon fiber, followed by a drying step to remove the water phase. The pH of the silver nitrate solution is maintained between the range of 3 and 8. The drying step is conducted at a temperature ranging from 25° C. to 150° C.

b. High Temperature Thermocracking

The silver-containing activated carbon fiber is placed in a high temperature furnace, with a controlled temperature ranging from 120° C. and 450° C. for 5 to 120 minutes, to break the silver on the surface of the activated carbon fiber into ultra fine metal particles. To avoid the oxidization or podzolization of the activated carbon fiber, it is preferred for the high temperature thermocracking to be conducted in a vacuum or in the presence of a protective air such as nitrogen.

c. Washing

The high-temperature thermocracked silver-containing activated carbon fiber is washed with water for 1 minute to 600 hours to remove the excess silver on the surface of the activated carbon fiber. This step is followed by baking the washed, activated carbon fiber to produce a silver-carrying activated carbon fiber.

The silver-carrying activated carbon fiber prepared according to the above process has a BET specific surface of above 400 m²/g, carbon content of above 50 wt %, silver content of above 0.001 wt % of the activated carbon fiber, and a density of above 1.8 g/m³. The activated carbon fiber made from a material selected from a group consisting of polypropylene nitrile fiber, cellulose fiber, bitumen fiber, phenolic fiber, and a combination thereof can be used in the above process.

The relevant details of the above preparation process can be found in Taiwan (ROC) Patent Publication No. 0059115 (TW0059115). Moreover, the preparation of a silver-carrying activated carbon fiber also can be found in JP 10-99678; T. A. Oya, T. Wakahara, and S. Yoshida, Carbon, 31, 1243-1247, 1993; Fu, R., H. Zeng, and Y. Lu, “Studies on the Mechanisms of the Reaction of Activated Carbon Fibers with Oxidants,” Carbon, 32(4), 593-598(1994); and Wang, Y. L., Y. Z. Wan, X. H. Dong, G. X. Cheng, H. M. Tao, and T. Y. Wen, “Preparation and Characterization of Antibacterial Viscose-based Activated Carbon Fiber Supporting Silver,” Carbon, 36(11), 1567-1571(1998). The above documents are incorporated hereinto for reference.

Manner II:

A triode electrochemical system is utilized, wherein the auxiliary electrode is titanium-plated platinum, the working electrode is graphite, and the reference electrode is reticular silver wires. The reference electrode is located above the working electrode. An electrolytic solution is formulated by dissolving AgNO₃ solids in water at a concentration of 0.001M. A graphite sheet, activated carbon fiber, carbon fiber, or graphite fiber is immersed in the electrolytic solution and is electroplated with an over-potential of −20 mV in a pulsed electroplating manner. After 5 to 100 cycles, the surface of the graphite sheet, activated carbon fiber, carbon fiber or graphite fiber is plated with silver particles with a grain size of no more than 200 nm.

Manner III:

A diode electrochemical system is utilized, where a graphite sheet is on both the cathode and anode. A graphite sheet, activated carbon fiber, carbon fiber, or graphite fiber is immersed in 0.001M of AgNO₃ electrolytic solution, and is electroplated with an electron current of −5 mA for 10 seconds. The surface of the graphite sheet, activated carbon fiber, carbon fiber or graphite fiber is plated with silver particles with a grain size of no more than 200 nm.

If the noble metal-carrying carbonaceous material used in the wound dressing of the present invention is in the form of fiber, it can be used as fiber, cloth, felt, ribbon or yarn. Optionally, woven fabrics (e.g., silver-carrying activated carbon fabrics) or non-woven fabric prepared from carbonaceous fibers or a fiber mixture containing carbonaceous fibers, or a carbon paper prepared from carbonaceous fibers or a fiber mixture containing carbonaceous fibers and an optional resin, can be directly used to provide the wound dressing of the present invention. It is then unnecessary to use a base layer. Moreover, the carbonaceous material can be blended into a polymeric material to provide one or more layers of the wound dressing like synthetic skin.

If the base layer of the wound dressing is a porous base, the noble metal-carrying carbonaceous material can be continuously or discontinuously coated on at least one portion of the first surface of the base. For example (but not limited to), the noble metal-carrying carbonaceous material can be mixed with a first adhesive component, so that the mixture is coated on the first surface of the mixture to enhance the bond between the carbonaceous material and the base. Therefore, if the mixture layer in the wound dressing comes into contact with the wound or the surrounding skin, an adhesive that does not stimulate the skin, preferably a pressure sensitive adhesive, is more suitable as the first adhesive. The first adhesive can also be coated on the first surface of the base, followed by applying the noble metal-carrying carbonaceous material on the adhesive. If the carbonaceous material is a carbonaceous fiber fabric, such as cloth, the carbonaceous fiber fabric layer and the base layer can be combined using ultrasonic welding to laminate the two layers, or simply by adding an additional adhesive layer to bind the two layers. As a result, the wound dressing is provided in the form of a band-aid.

If the base layer of the wound dressing is drug-impermeable, the noble metal-carrying carbonaceous material can be directly applied onto at least one portion of the first surface of the base as a material layer of carbonaceous material. In this respect, if the carbonaceous material is a carbonaceous fiber, the carbonaceous fiber can be applied onto at least one portion of the first surface of the base as a fabric layer. The carbonaceous fabric layer can be laminated to the base layer using ultrasonic welding. In this case, the carbonaceous fabric layer is composed of carbonaceous fiber and natural and/or synthetic fiber. It can also contain just the carbonaceous fiber. Optionally, an additional adhesive layer can be used between the first surface of the base and the carbonaceous material layer to bind the carbonaceous material layer and the first surface. Alternatively, the carbonaceous material layer can directly contain an adhesive. In this case, if the carbonaceous material layer in a wound dressing comes into contact with the wound or the surrounding skin, an adhesive that does not stimulate the skin, preferably a pressure sensitive adhesive, is more suitable.

Pressure sensitive adhesives that are known to be commonly applied in wound dressings can be utilized in the present invention. For example, the pressure sensitive adhesives disclosed in U.S. Pat. No. 4,675,009, U.S. Pat. No. 4,696,854, U.S. Pat. No. 5,536,263, U.S. Pat. No. 5,741,510, U.S. Pat. No. 5,972,377, and U.S. Pat. No. 6,495,158 B1 can be used. The above patents are incorporated hereinto for reference. The pressure sensitive adhesives useful in the present invention include, but not limited to, the following pressure sensitive adhesives for use in a percutaneous patch: polyacrylate, siloxane, or polyisobutadiene.

The wound dressing of the present invention can optionally comprise other therapeutically active components to provide additional therapeutic benefits. The therapeutically active component can either be incorporated into the layer with the noble metal-carrying carbonaceous material, or exist individually in a separate layer. If the therapeutically active component and the carbonaceous material individually exist in separate drug layers, the layer containing the carbonaceous material may be placed between the base layer and the drug layer. In this case, the base layer can be, as mentioned above, a porous base and the carbonaceous material can be coated onto the base. Moreover, the drug layer can further comprise a pressure sensitive adhesive component suitable for a percutaneous patch. Alternatively, the carbonaceous material layer can be inserted in between the two drug layers (i.e., base layer, drug layer, carbonaceous material layer, and drug layer in order). The therapeutically active components and pressure sensitive adhesive components contained in the drug layers can either be the same or different.

Any therapeutically active component for healing skin wounds can be applied in the wound dressing of the present invention. For example (but not limited to), the therapeutically active component useful in the wound dressing of the present invention comprises an antifungal agent such as erythromycin, tetracycline, clindamycin, cephalosporin, acrisorcin, haloprogin, iodochlorhydroxyquin, tolnaftate, and triacetin, as well as a drug component for trauma, burn, and scald such as centella asiatica, econazole nitrate, mafenide, mupirocin, and povidone iodine.

Optionally, the wound dressing of the present invention can further comprise a protective layer on the exterior surface of the material layer which contains a pressure sensitive adhesive (the material layer can be a drug layer, a carbonaceous material layer, or a separate adhesive layer) to protect the material. The protective layer can be a single material layer or two material layers which partially overlap each other. Any known protective layers useful in wound dressings can be used in the present invention. For example (but not limited to), a film consisting of one or more the following materials can be used as the protective layer: polyurethane, nylon, polyamide, polycellulose, polyvinyl chloride, polyvinyl dichloride, polyolefin (e.g., ethylene vinylacetate copolymer, polyethylene, and polypropylene), polyurea, and polyester (e.g., polyethylene terephthlate).

The known technology used in the wound dressing of the present invention is briefly mentioned to facilitate the explication of the present invention. The technical features and contents of the present invention will be further explained according to the drawings. Nonetheless, the relevant drawings are not drawn according to the actual proportion, since they function to express the features of the present invention only.

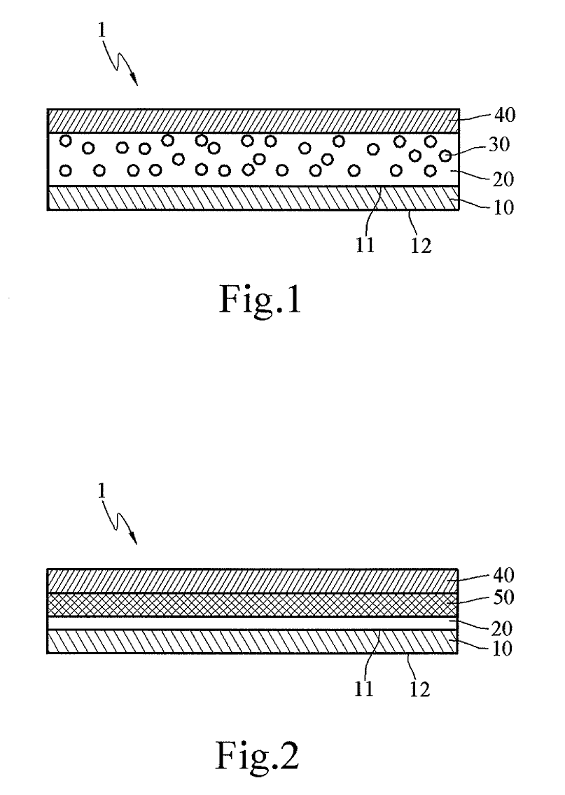

FIG. 1 shows a side view of an embodiment of the wound dressing of the present invention, wherein the wound dressing is a patch (1) for healing a wound. The patch (1) contains a base layer (10) with a first surface (11) and a second surface (12), an antimicrobial layer (20) on said first surface (11), and a protective layer (40) on said antimicrobial layer (20). The antimicrobial layer (20) comprises a pressure sensitive adhesive (not depicted) and a silver-carrying carbonaceous material (30), as well as an optional therapeutically active component (not depicted) for healing trauma, burn, or scald wounds. The carbonaceous material can be activated carbon, graphite, carbon, or a combination thereof; preferably, activated carbon, porous graphite, or a combination thereof. Some examples include (but not limited to): activated carbon fiber, powder or particulate charcoal, and/or porous graphite such as flexible graphite or extended graphite. When using the patch (1), the protective layer (40) is torn from said antimicrobial layer (20) and then the patch (1) is applied onto the wound or injured portion of the body with the side containing the antimicrobial layer (20).

FIG. 2 shows a side view of another embodiment of the wound dressing of the present invention. The schematic patch (1) contains a base layer (10) with a first surface (11) and a second surface (12), an antimicrobial layer (20) on said first surface (11), a drug layer (50) coated on said antimicrobial layer (20), and a protective layer (40) on said drug layer (50). The drug layer (50) contains a pressure sensitive adhesive (not depicted) and a therapeutically active component (not depicted) for healing burn and scald wounds. The antimicrobial layer (20) is composed of a silver-carrying carbonaceous material and may also have an adhesive. The carbonaceous material can be activated carbon, graphite, carbon, or a combination thereof; preferably, activated carbon, porous graphite, or a combination thereof. Some examples include(but not limited to): activated carbon fiber, powder or particulate charcoal, and/or porous graphite such as flexible graphite or extended graphite. When using the patch (1), the protective layer (40) is torn from said drug layer (50) and then the patch (1) is applied onto the wound or injured portion of the body with the side containing the drug layer (50). Optionally, another drug layer is placed between the base layer (10) and the antimicrobial layer (20).

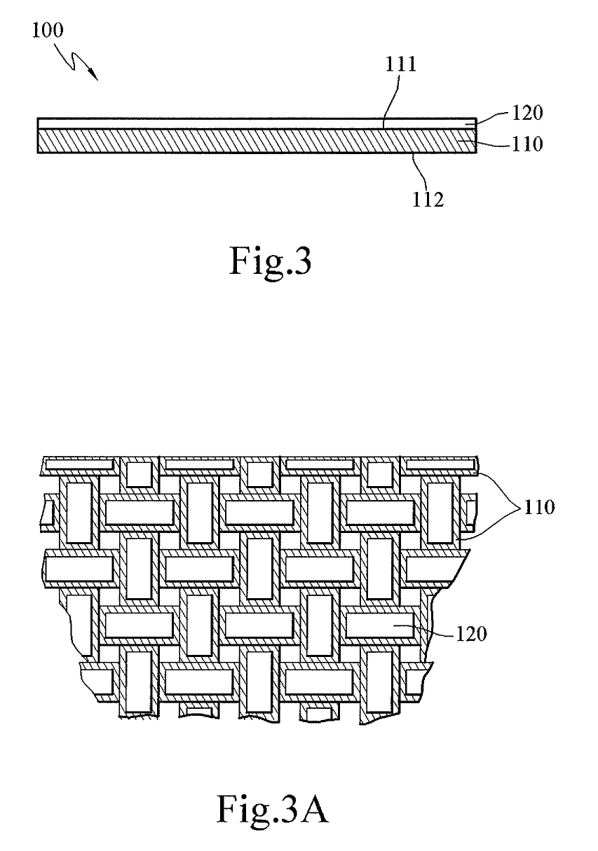

FIG. 3 shows a side view of another embodiment of the wound dressing of the present invention, wherein the wound dressing is a gauze (100). FIG. 3A is a partially enlarged top view of the gauze (100). As depicted in FIGS. 3 and 3A, the gauze (100) contains a fiber fabric base layer (110) that has a first surface (111), a second surface (112) and an antimicrobial coating (120) applied onto at least one portion of said first surface (111). The antimicrobial coating (120) contains a silver-carrying carbonaceous material (not depicted) and a pressure sensitive adhesive (not depicted). The carbonaceous material can be activated carbon, graphite, carbon, or a combination thereof; preferably activated carbon, porous graphite, or a combination thereof. Some examples include (but not limited to): activated carbon fiber, powder or particulate charcoal, and/or porous graphite such as flexible graphite or extended graphite. In this embodiment, when the silver-carrying carbonaceous material is carbonaceous fiber, the carbonaceous fiber can be directly incorporated into the fiber of the base layer (110). The carbonaceous fiber can also be directly used as the base layer (110), making it unnecessary to have an adhesive or even an additional fiber layer.

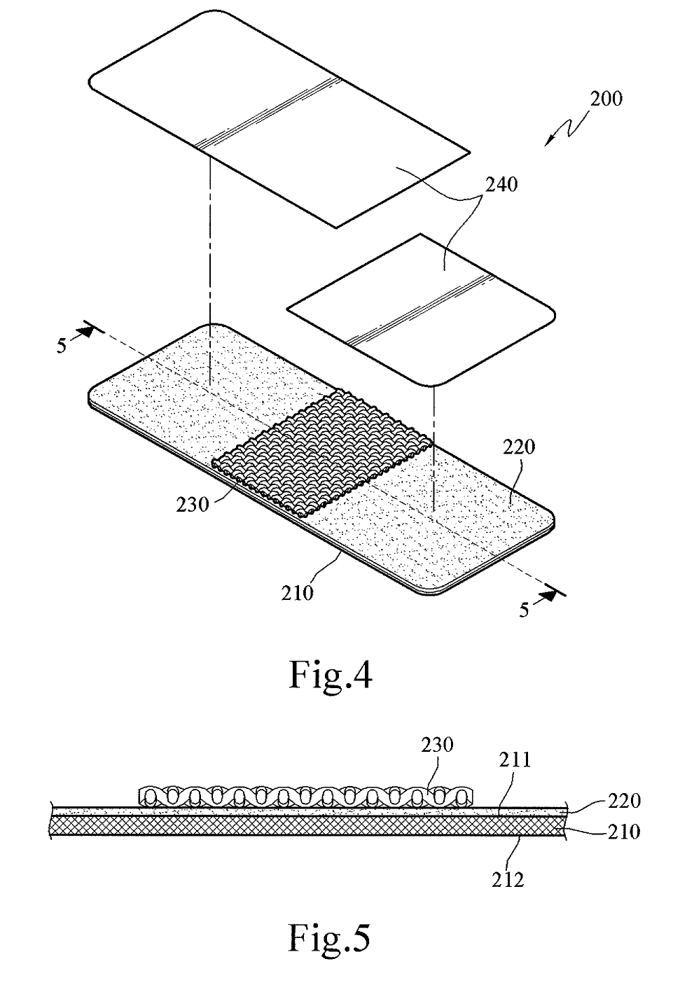

FIG. 4 shows a schematic diagram of another embodiment of the wound dressing of the present invention, wherein the wound dressing is a band-aid (200). FIG. 5 shows a cross-sectional view along line 5-5 of FIG. 4. As depicted in FIGS. 4 and 5, the band-aid (200) contains a base layer (210) that has a first surface (211) and a second surface (212), a pressure sensitive adhesive (220) coated on said first surface (211), an antimicrobial fabric layer (230) preferably positioned on the center of the base layer (210), and two protective layers (240) which partially overlap each other. The antimicrobial fabric layer (230) is a gauze layer comprising a silver-carrying carbonaceous fiber, preferably, silver-carrying activated carbon fiber. Although the antimicrobial layer (230) is depicted by a gauze, the antimicrobial layer (230) can also be a flexible carbonaceous material sheet or a non-woven fabric or fiber paper containing silver-carrying carbonaceous fiber. Optionally, a drug layer (not depicted) with another drug component for healing trauma, burn, or scald wounds can be applied onto the antimicrobial layer (230) to provide additional therapeutic effects.

In the wound dressing of the present invention where the noble metal is carried on the activated carbon or porous graphite, the activated carbon/porous graphite actively absorbs and aggregates the microbes close to the wound dressing on its surface. The activated carbon/porous graphite-carried noble metal proceeds to destroy the absorbed/aggregated microbes to provide an aseptic condition for the wound, promote the wound occlusion, and reduce scarring. Moreover, in the case where the activated carbon is used to carry the noble metal, moisture does not need to be added to activate the noble metal because the activated carbon absorbs moisture itself (normally 5 to 20 wt %). With this simpler manner, the present invention is still able to attain the desired antimicrobial benefit.

The present invention also provides a kit comprising the wound dressing of the subject invention and a fixing element. Any device that can locally fix the dressing can be used as the fixing element. For example (but not limited to), the fixing element could be a tape, a bandage, or a patch.

The present invention further provides an antimicrobial composition for topical use on skin. The composition comprises a carbonaceous material and an effective amount of noble metal carried on the carbonaceous material. The carbonaceous material is selected from a group consisting of activated carbon, graphite, carbon, or a combination thereof; preferably, activated carbon, porous graphite, or a combination thereof. Some examples include (but not limited to): activated carbon fiber, powder or particulate charcoal, and/or porous graphite, such as flexible graphite or extended graphite. The noble metal is selected from a group consisting of silver, gold, palladium, platinum, copper, zinc, and a combination thereof. Preferably, the noble metal is silver.

In the composition of the present invention, the amount of the noble metal carried on the carbonaceous material is not critical, as long as its amount can provide the desired antimicrobial benefits. In view of the weight of the noble metal per se, it is typically 0.0001 to 30 wt %, preferably, 0.001 to 10 wt % of the total weight of the composition. It is preferred that the noble metal is carried on a carbonaceous material in particulate form. To more effectively release the noble metal ions that have antimicrobial activity, it is more preferred for the carbonaceous material-carried particulate noble metal to have a grain size of no more than 200 run, more preferably, no more than 100 nm, and most preferably, no more than 50 nm.

Optionally, the composition of the present invention further contains a therapeutically active component in a therapeutically effective amount. For example (but not limited to), the therapeutically active components comprise an antimicrobial agent, an antifungal agent, or other therapeutic components for trauma, burn, or scald wounds. For example (but not limited to), the composition of the present invention can optionally contain erythromycin, tetracycline, clindamycion, cephalosporin, triclosan, phenoxy isopropanol, chlorhexidine gludonate, povidone iodine, acrisorcin, haloprogin, iodochlorhydroxyquin, tolnaftate, triacetin, centella asiatica, econazole nitrate, mafenide, mupirocin, and/or povidone iodine.

In addition to the active components, the composition of the present invention can further contain a non-toxic, pharmaceutically and skin acceptable carrier, diluent, and excipient suitable for topical use. The carrier, diluent, and excipient, and the standard doses of known pharmacological agents can be found in U.S. Pat. No. 6,692,773 B2. The contents are incorporated hereinto for reference.

The composition for topical use of the present invention can be in various dosage forms, such as a gel, a paste, an ointment, a cream, an emulsion, or a suspension. The noble metal-carrying carbonaceous material can be mixed with the pharmaceutically acceptable carrier, diluent, or excipient and other optional active components under aseptic conditions to provide the desired dosage form. For example (but not limited to), a suitable thickener or gelling agent is added to an aqueous or oil base to formulate a facial mask, ointment, or cream. Water can be used as the aqueous base. Depending upon the inherent properties of the base, aluminum stearate and hydrogenated lanolin can be used as a thickener. Starch, tragacanth, cellulose derivative, polyethylene glycol, silicones, bentonite, silicic acid, talc, or a mixture thereof can be used as the excipient to provide the composition of the present invention in a dosage form of a paste, an ointment, a cream, or a gel.

It should be noted that the major difference between the subject invention and the prior art lies in that the noble metal, which has an antimicrobial characteristic, is used in the present invention in the form of a carbonaceous material-carried noble metal. With the use of activated carbon because of its ability to absorb moisture itself, the composition of the present invention can activate the noble metal to provide the antimicrobial benefit without adding moisture. Moreover, using activated carbon/porous graphite as the carbonaceous material due to its ability to aggregate microbes, the present invention improves the “passive” characteristic presented by the prior art, in which the noble metal only kills the microbes upon contact. That is, the present invention actively destroys microbes. Therefore, the present invention is suitable for use in situations known in the prior art where the noble metals are topically used on skins to destroy microbes and inhibit their growth, so as to provide a superior antimicrobial benefit.

Although the present invention has been disclosed above, the disclosure does not limit the present invention. Persons having ordinary skill in the art can make any changes or modifications without departing from the spirit and scope of the present invention. Consequently, the scope of protection of the present invention is based on the claims attached.

What is claimed is:

1. A wound dressing, comprising a carbonaceous material and a noble metal carried on the carbonaceous material, wherein said carbonaceous material is selected from a group consisting of activated carbon, graphite, carbon, and a combination thereof, and said noble metal is selected from a group consisting of silver, gold, palladium, platinum, copper, zinc, and a combination thereof.

2. The dressing according to claim 1, wherein the carbonaceous material is selected from a group consisting of activated carbon, porous graphite, and a combination thereof.

3. The dressing according to claim 2, wherein the porous graphite is selected from a group consisting of flexible graphite, extended graphite, and a combination thereof.

4. The dressing according to claim 1, wherein said noble metal is silver and has a grain size of no more than 200 nm.

5. The dressing according to claim 1, further comprising a flexible base layer having a first surface and a second surface, wherein said noble metal-carrying carbonaceous material is coated on at least one portion of said first surface.

6. The dressing according to claim 5, wherein the noble metal-carrying carbonaceous material is attached to the first surface with a first adhesive.

7. The dressing according to claim 5, further comprising a second adhesive coated on the carbonaceous material.

8. The dressing according to claim 6, further comprising a second adhesive coated on the carbonaceous material.

9. The dressing according to claim 1, further comprising a therapeutically active component.

10. The dressing according to claim 9, wherein said therapeutically active component is selected from a group consisting of erythromycin, tetracycline, clindamycin, cephalosporin, acrisorcin, haloprogin, iodochlorhydroxyquin, tolnaftate, triacetin, centella asiatica, econazole nitrate, mafenide, mupirocin, povidone iodine, and a combination thereof

11. The dressing according to claim 1, wherein the dressing is woven fabric, non-woven fabric, or synthetic skin.

12. The dressing according to claim 1, wherein the carbonaceous material is in a form selected from a group consisting of powder, particle, fiber, sheet, and a combination thereof.

13. A kit comprising a wound dressing according to claim 1 and a fixing element.

14. The kit according to claim 13, wherein the fixing element is a tape, a bandage or a patch.

15. An antimicrobial composition for topical use on skins, comprising a carbonaceous material and an effective amount of noble metal carried on the carbonaceous material, wherein said carbonaceous material is selected from a group consisting of activated carbon, graphite, carbon, and a combination thereof, and said noble metal is selected from a group consisting of silver, gold, palladium, platinum, copper, zinc, and a combination thereof.

16. The antimicrobial composition according to claim 15, wherein the carbonaceous material is selected from a group consisting of activated carbon, porous graphite, and a combination thereof.

17. The antimicrobial composition according to claim 16, wherein the porous graphite is selected from a group consisting of flexible graphite, extended graphite, and a combination thereof.

18. The antimicrobial composition according to claim 17, wherein said noble metal is silver and has a grain size of no more than 200 nm.

19. The antimicrobial composition according to claim 15, further comprising a therapeutically active component.

20. The antimicrobial composition according to claim 19, wherein the therapeutically active component is selected from a group consisting of erythromycin, tetracycline, clindamycin, cephalosporin, acrisorcin, haloprogin, iodochlorhydroxyquin, tolnaftata, triacetin, centella asiatica, econazole nitrate, mafenide, mupirocin, povidone iodine, and a combination thereof.

21. The antimicrobial composition according to claim 20, for use in a facial mask.
